(19) United States
(12) Patent Application Publication
(10) Pub. No.: US 2025/0067731 A1
(43) Pub. Date: Feb. 27, 2025

(54) SEROLOGICAL ASSAYS FOR PARKINSON'S DISEASE

(71) Applicant: NeuroDex, Inc., Natick, MA (US)

(72) Inventors: Erez Eitan, Arlington, MA (US); Olga Volpert, Waltham, MA (US)

(21) Appl. No.: 18/720,620

(22) PCT Filed: Dec. 19, 2022
(86) PCT No.: PCT/IB2022/062455

§ 371 Date: Jun. 14, 2024

Related U.S. Application Data
(60) Provisional application No. 63/291,405, filed on Dec. 19, 2021.
(60) Provisional application No. 63/291,407, filed on Dec. 19, 2021.

(51) Int. Cl. G01N 33/543; G01N 33/58; G01N 33/68

(52) U.S. Cl. CPC G01N 33/54333; G01N 33/582; G01N 33/6896; G01N 2333/46; G01N 2800/2821; G01N 2800/2835; G01N 2800/285; G01N 2800/52

(57) ABSTRACT

The present invention provides improved and minimally invasive biomarker-based diagnostics for synucleinopathies (e.g., Parkinson's disease (PD)). The invention further provides assays and methods for analysis of biological samples, for the evaluation and determination of characteristics pertaining to pathological processes associated with α-synuclein, and to methods for determining the suitability of analytical agents for diagnosis of synucleinopathies. More specifically, the invention in embodiments thereof provides improved methods comprising specific detection and quantification of α-synuclein-based biomarkers from low volumes of biological samples.

### FIELD OF THE INVENTION

The present invention relates to improved and minimally invasive biomarker-based diagnostics for Parkinson's disease (PD) and other synucleinopathies.

### BACKGROUND OF THE INVENTION

Parkinson's disease (PD) is a progressive degenerative disorder of the central nervous system, characterized by a loss of dopaminergic neurons in the brain's substantia nigra and extensive accumulation of α-synuclein aggregates in the form of inflammation-inducing Lewy bodies. It manifests as a syndrome with severe motor and cognitive symptoms and a grim prognosis, compounded by an absence of definitive diagnostic measures. To date, there are no accepted biomarkers used in clinical practice to unequivocally diagnose PD and effectively differentiate it from other neurodegenerative diseases with a similar pathophysiology. Disease onset is thought to occur after a devastating and irretrievable loss of about 70-80% of dopaminergic neurons, with corroboration possible only post-mortem. To aid in clinical management and hopefully ameliorate patients' suffering before it begins, a method for early detection of PD's pathophysiological processes is critically needed to protect against the destructive onset of Parkinsonism.

Currently acceptable diagnostic measures rely solely on clinical characteristics, greatly hampering prognostic success due to late-stage detection, when preventative measures are no longer feasible, and the accuracy of such diagnoses remains unsatisfactory. Attempts have been made to identify suitable biomarkers for reliable PD diagnosis, with α-synuclein species, lysosomal enzymes, markers of amyloid and tau pathology, and neurofilament light chain being of particular interest. While total α-synuclein levels in cerebrospinal fluid (CSF) and blood per se exhibit low diagnostic accuracy and are highly prone to contamination by erythrocytes, the combination of several CSF markers, including α-synuclein species, was suggested to improve the diagnostic and prognostic value of the assay, and in particular to differentiate PD patients from patients with other neurological disorders (Parnetti et al. 2019, The Lancet Neurology, Vol. 18, Issue 6, 573-586).

Additional disorders in which accumulation or dysregulation of α-synuclein is implicated in their etiology and/or pathology, collectively termed synucleinopathies, include for example Lewy body dementia (LBD), PD with dementia (PDD) pure autonomic failure (PAF), and multiple system atrophy (MSA). These disorders may be characterized by overlapping symptoms or features. In addition, abnormal deposition of α-synuclein may also be observed in brains of patients with neurodegenerative disorders which represent a population suffering from mixed pathology.

A new avenue of biomarker identification has opened with the advent of extracellular vesicle isolation and characterization. The umbrella term “extracellular vesicles” (EV) refers to a heterogeneous array of secreted membrane vacuoles released from all cell types, carrying a molecular cache that may give an indication as to the contents of the parent cell. Exosomes are a subtype of EV, initially believed to carry only cellular waste, now widely accepted as being an important aspect of cell-to-cell communication. Due to their potential to carry cell-specific cargo and their secretion from all cell types to a detectable level in many bodily fluids, exosomes have garnered increasing interest as to their clinical potential in biomarker-based diagnostics and drug delivery. For example, EV-based biomarkers and their potential use are disclosed in U.S. Pat. No. 9,958,460 and WO2017193115. Various other publications involving the isolation and/or analysis of vesicles from biological samples include e.g. US20190219578, US20180340945, US20190361037, US20180080945, US20190137517, WO2016172598 and WO2021231720. Potential therapeutic uses of EV are disclosed in U.S. Pat. No. 11,111,475.

As α-synuclein was also found in lysates of certain EV populations, attempts have been made to examine the potential use of α-synuclein-bearing EV in PD diagnosis. However, while some reports identified differences between PD patients and healthy controls, these differences were mostly non-significant or inconsistent. In order to enhance the sensitivity of detection and the diagnostic accuracy, additional steps for EV enrichment (such as immunoprecipitation) and/or the use of additional diagnostic markers were suggested.

For example, Jiang et al. examined the levels of α-synuclein in lysates of neuronal exosomes isolated from serum samples (using L1CAM-specific antibodies). The publication discloses that exosomal α-synuclein per se was not sufficiently sensitive and specific to be used as a diagnostic marker, and suggests a combination of this analyte with exosomal clusterin as a measure to predict and differentiate PD from atypical parkinsonism (Jiang et al. 2020. J Neurol Neurosurg Psychiatry, 91:720-729). In a subsequent publication (Dutta et al. 2021. Acta Neuropathologica 142:495-511), α-synuclein levels measured in lysates of blood exosomes immunoprecipitated using neuronal and oligodendroglial markers (L1CAM and or MOG, respectively), were found to distinguish PD from multiple system atrophy (MSA). However, the study indicates that the separation of patients with PD from healthy individuals was not clinically sufficient, in line with previous publications, and the authors also suggest the addition of biomarkers, such as tau and clusterin to improve the diagnostic accuracy.

WO2021094751 relates to using α-synuclein and clusterin as measured in exosomes isolated from serum, as biomarkers in the prediction and identification of a subject having PD, and provides methods for determining their levels. The biomarkers are also disclosed to be useful for monitoring, prevention and/or treatment of PD and in differentiating PD from atypical parkinsonian syndromes including MSA.

WO2017032871 provides methods of differential diagnosis of dementia with Lewy bodies and PD, comprising a step of isolating exosomes from a CSF sample, and determining the number of exosomes in a defined volume of CSF sample and/or the amount of α-synuclein within said exosomes.

WO2019153748 discloses methods of enriching or detecting astrocytic exosomes by creating an immune complex with anti-GLT1 antibodies, for the purpose of auxiliary diagnoses, differential diagnoses and monitoring central nervous system diseases. The publication further suggests measurement of various biomarkers, including α-synuclein or phosphorylated α-synuclein, in the isolated exosomes, when the subject is suspected of having a neurological disease such as PD.

WO2019161302 relates to methods for assessing the likelihood for developing PD, comprising measuring in a tear or saliva sample isolated from the subject, the level or activity of at least one biomarker of PD, inter alia α-synuclein, which may optionally be in phosphorylated or oligomerized forms. The publication further suggests that measuring the level or activity of biomarkers may be performed using certain unspecified exosomes that may be isolated from the tear or saliva sample. The publication further teaches that methods for isolating or enriching exosomes include column chromatography, differential centrifugation, and/or nanoparticle tracking.

However, to date, no serological assay for PD and other synucleinopathies is available for clinical use, and diagnosis remains error-prone and cumbersome. There is a long-felt need in the art for a minimally invasive, sensitive, and accurate assay for adequate and early assessment and diagnosis of synucleinopathies, which could aid in clinical management and increase prognostic success.

### SUMMARY OF THE INVENTION

The present invention relates to improved and minimally invasive biomarker-based diagnostics for synucleinopathies (e.g., Parkinson's disease (PD)). The invention further provides assays and methods for analyzing biological samples for the evaluation and determination of characteristics pertaining to pathological processes associated with α-synuclein, and to methods for determining the compatibility of analytical agents with diagnosis of synucleinopathies. More specifically, the invention in embodiments thereof relates to improved methods comprising quantification of α-synuclein-based biomarkers on the surface of extracellular vesicles (EV).

The invention is based, in part, on the development of an unexpectedly improved assay for detecting and analyzing EV-associated biomarkers, providing for specific quantification of α-synuclein (αSyn) forms in low volume plasma samples. In particular, an assay was developed in which intact EV were captured from biological samples using distinct color-coded magnetic microspheres, and simultaneously analyzed, using Luminex technology, for the level of αSyn bound to the surface of EV from different cellular sources.

Unexpectedly, using the assays developed and disclosed herein, it was discovered that αSyn-specific antibodies differ in their ability to identify αSyn on the surface of exosomes from different cells of origin and in their ability to differentiate PD patients from healthy controls. In particular, the ability to detect αSyn on erythrocyte EV was surprisingly found to be associated with poor diagnostic capacity, whereas selectivity towards αSyn forms presented on neural and glial EV populations was correlated with enhanced diagnostic capacity. The invention is further based, in part, on the discovery of advantageous and improved diagnostic assays for synucleinopathies, providing unexpectedly high accuracy in differentiating subjects with a synucleinopathy (e.g., PD patients, LBD patients) from healthy controls in a minimally invasive manner, using low plasma sample input (<50 μl volume) per assay data point.

Accordingly, disclosed herein are methods and assays for the detection and diagnosis of a synucleinopathy (e.g., PD), and for evaluation of pathological processes associated with α-synuclein proteinopathy (synucleinopathy).

The assays and methods of the invention involve the specific detection of membrane-bound α-synuclein on certain EV populations. In particular, assays and methods according to the principles of the invention comprise selective assessment or quantification of α-synuclein that had bound to the surface of EV in a differential, tissue-specific manner. In other words, the methods and systems disclosed herein differentiate between intra-exosomal α-synuclein and α-synuclein exposed on the EV outer membranes (the latter designated herein “membrane-bound αSyn” or “surface-bound αSyn”), and between α-synuclein forms associated with EV derived from neurons (or other cells of the nervous system such as glial cells), and α-synuclein forms associated with other cell types, such as erythrocytes. The invention in advantageous embodiments thereof further relates to multiplexed assays enabling enhanced diagnostic capacities.

Due to the unique characteristics disclosed herein, including in particular, the detection of membrane-bound αSyn in different cell type-derived EV with high precision and specificity, tissue selectivity and enhanced detection capacities, the assays and methods of the invention overcome disadvantages of hitherto disclosed αSyn-based assays, e.g. inadequately low diagnostic accuracy and reproducibility and susceptibility to contamination by erythrocytes. The assays and methods of the invention can thus be performed with high fidelity in a minimally invasive manner using low volumes of blood-derived samples.

Thus, according to a first aspect of the invention, there is provided a method of determining the presence or absence of a synucleinopathy in a subject in need thereof, comprising selectively assessing in a biofluid sample of the subject, the level of membrane-bound αSyn, specifically on the surface of at least one EV population of a nervous system origin.

As used herein, the term “membrane-bound αSyn” is distinguished from “total αSyn” or “intra-vesicular αSyn”, as will be discussed in further detail below. In the context of the present invention, “selective quantification (or assessment) of membrane-bound αSyn” denotes that the quantified levels reflect those measurable on intact EV of the particular cellular origin, and do not reflect intra-vesicular αSyn levels (such as those measurable in EV subjected to lysis or permeabilization). Further, as disclosed herein, selective assessment of membrane-bound αSyn specifically on an EV population of a particular cellular origin (such as of neural or glial origin, e.g. neuron, oligodendrocyte or microglia-derived EV), indicates that αSyn levels of other EV populations, and in particular of erythrocyte-derived EV (EDE), are below detection limit, and do not significantly affect the assessment.

In various embodiments, said at least one EV population is selected from the group consisting of neural-derived EV (NDE), oligodendrocyte-derived EV (ODE), and microglia-derived EV (MDE). In a particular embodiment, said EV population is MDE. In some embodiments, the methods of the invention advantageously include selectively assessing (e.g. quantifying) membrane-bound αSyn on the surface of two or more of said EV populations (thereby providing a separate assessment or quantification corresponding to each of the two or more EV populations). In another particular embodiment, said two or more of said EV populations comprise MDE. In yet another particular embodiment, said EV populations are ODE and MDE. In yet another particular embodiment, said EV populations comprise ODE and MDE. In a further particular embodiment, said EV populations are NDE, ODE and MDE. In a further particular embodiment, said EV populations comprise NDE, ODE and MDE. In another embodiment, said at least one EV populations of a nervous system origin is characterized by an average particle size of 50-120 nm. In another embodiment, said at least one EV populations of a nervous system origin is characterized by an average particle size of 10-30 nm. In yet another embodiment, said at least one EV populations of a nervous system origin comprises a first population characterized by an average particle size of 10-30 nm and a second population characterized by an average particle size of 50-120 nm. Each possibility represents a separate embodiment of the invention. Advantageously, the αSyn levels in multiple EV populations are determined and assessed simultaneously, from the same sample, so as to provide specific measurements of the quantified αSyn levels corresponding to each EV population in a single measurement, as will be explained in further detail below. In another embodiment, the αSyn is phosphorylated (e.g. on serine 129). In another embodiment, the αSyn is non-phosphorylated. In another embodiment, the measured αSyn level includes the levels of both phosphorylated and non-phosphorylated αSyn. In another embodiment, the method comprises specific assessment of an αSyn form that is associated with (or detectable on) EV populations of a nervous system origin, and is not substantially associated with (or detectable on) EDE.

In another embodiment, the measured αSyn levels are normalized, e.g. by a value. In a further particular embodiment, said membrane-bound αSyn levels assessed on EV populations comprising NDE, ODE and MDE are normalized by division by general EV marker CD63 levels on NDE, ODE and MDE, respectively. In a further particular embodiment, said membrane-bound αSyn levels assessed on EV populations comprising NDE, ODE and MDE are normalized by division by general EV marker CD81 on NDE, ODE and MDE, respectively. In a further particular embodiment, said membrane-bound αSyn levels assessed on EV populations comprising NDE, ODE and MDE are normalized by division by general EV marker CD9 on NDE, ODE and MDE, respectively.

In another embodiment, the method further comprises selectively assessing the level of membrane-bound αSyn specifically on the surface of at least one EV population of a nervous system (e.g. neural or glial) origin in a sample of a healthy control individual. In another embodiment, an αSyn level in the sample of the subject that is significantly higher than the level assessed in the control sample, indicates the presence of a synucleinopathy in said subject. In another embodiment, an αSyn level in the sample of the subject that is not substantially higher than the level assessed in the control sample, indicates the absence of a synucleinopathy in said subject.

In another embodiment (e.g. when αSyn levels in multiple EV populations is determined), the method considers the levels measured in each of the EV populations, in order to determine the presence or absence of a synucleinopathy. For example, in some embodiments, the method comprises comparing the levels of αSyn as assessed in each EV population to their respective levels corresponding to a control sample, to thereby compare the diagnostic signature of the sample to the control diagnostic signature, wherein a significant difference in the diagnostic signature of the subject compared to the control diagnostic signature indicates that said subject is afflicted with a synucleinopathy. In another embodiment, a diagnostic signature reflecting levels of αSyn in the EV populations of the subject that are significantly higher than the levels assessed in a control sample corresponding to a healthy control subject, indicates the presence of a synucleinopathy in said subject.

In another embodiment, the method may further comprise determining the levels of additional biomarkers. However, as disclosed herein, the methods and assays of the invention are sufficiently accurate to provide for diagnosis of the presence of a synucleinopathy without the use of other, unrelated biomarkers (such as clusterin). Thus, in other embodiments, quantification of clusterin levels is explicitly excluded. In another embodiment, the method comprises determination of membrane-bound αSyn levels as disclosed herein as the sole diagnostic marker.

In another embodiment, said αSyn is quantified using a Luminex-based assay. Typically and advantageously, the assay is employed (or assessment or quantification is performed) under conditions such that the EV remain substantially intact. For example, without limitation, the assays and methods of the invention advantageously employ the use of detergent-free buffers (e.g. when incubating a sample with a capture system in accordance with the invention) which may comprise protease and/or phosphatase inhibitors. In additional advantageous embodiments, assays and methods of the invention may involve enhancement of the salt concentration during the washing steps, while maintaining an essentially detergent-free environment. In another embodiment, said αSyn is assessed by a method or assay as disclosed herein.

In some embodiments the invention employs high salt conditions (e.g. 50 mM-300 mM NaCl) to reduce non-specific interactions and improve the specificity of the measurement in complex blood-based biofluids. In some embodiments the invention employs a blocking strategy comprising competition with negatively charged peptides that reduces the interactions of the overall negatively charged EVs, and thus improves the specificity of the measurement in a complex blood-based biofluid.

Biofluid samples used in connection with the methods and assays of the invention comprise intact EV. In some embodiments, the sample is a blood-derived sample (e.g. a plasma or serum sample). Advantageously, as demonstrated herein, the method is amenable for use with low input volumes of biofluid samples, e.g. 1-100 μl, less than 75 μl per data point (measurement) and typically 1-75 μl or 1-50 μl of plasma or serum samples. In another embodiment, the sample comprises 1-25, 15-25, or 10-20 μl (which may be diluted to a final volume compatible with the chosen assay, for example about 50 μl for Luminex-based assays), wherein each possibility represents a separate embodiment of the invention. In another embodiment, the sample is obtained from a subject suspected of having a synucleinopathy.

In an exemplary embodiment, methods in accordance with the invention may comprise:
a. providing a system for EV capture, comprising a plurality (e.g. at least three populations) of distinct fluorescence-labeled magnetic microspheres, wherein each microsphere population displays antibodies directed to distinct targets on the surface of distinct EV populations (typically neural or glial populations, e.g. PLP1 for oligodendrocytes, P2RY12 for microglia and GAP43 for neurons);
b. providing a blood-derived sample of the subject (in particular-providing a plasma or serum sample), the sample comprising less than 75 μl (e.g., 1-75 μl) of non-processed plasma, or a corresponding amount of intact EV;
c. incubating the sample with the system, under conditions such as to allow specific antigen-antibody binding while substantially maintaining the integrity of the EV membranes, to thereby provide distinct populations of EV complexes with the microspheres (herein designated “EV-microsphere complexes”) corresponding to each target;
d. washing the EV-microsphere complexes using a magnetic device, under conditions enabling selective capturing of said complexes (e.g. to remove non-specific EV and soluble proteins);
e. incubating the EV-microsphere complexes with at least one labeled detection antibody (for example an antibody directed to αSyn and/or an antibody directed to S129P-αSyn), under conditions such as to allow specific antigen-antibody binding while substantially maintaining the integrity of the EV membranes;
f. optionally and advantageously washing the resulting labeled complexes using a magnetic device to remove excess reagents (e.g. unbound antibody);
g. subjecting the resulting complexes to a microfluidic device amenable for simultaneously detecting and quantifying fluorescent emission on a plurality of wave lengths, to thereby quantify the fluorescence emission levels and provide a separate assessment of the α-synuclein level corresponding to each of the EV populations; and;
h. comparing the quantified levels to control levels (while typically considering the background levels of each measurement).

For example, providing the separate assessment of the α-synuclein level corresponding to each of the EV populations may be performed by generating gates encompassing the fluorescence range of each microsphere type corresponding to each distinct EV population and quantifying the fluorescence emission levels corresponding to the detection antibody for data points within each gate.

In another embodiment, the at least one labeled detection antibody is directed to αSyn. In another embodiment, the at least one labeled detection antibody is directed to p-αSyn. In another embodiment, at least one additional labeled detection antibody may be used, directed to a general EV marker, e.g. CD63 and/or CD81. In yet another embodiment, the method does not include the use of additional detection antibodies such as antibodies directed to general exosomal markers. In another embodiment, the ratio between the αSyn and/or the p-αSyn levels in specific EV populations is determined. In another embodiment, said at least one labeled detection antibody is capable of selectively identifying αSyn on EV populations of a neural or glial origin, and not on EDE. In another embodiment said antibody is capable of selectively identifying an αSyn form that is associated with EV populations of a neural or glial origin, and is not substantially associated with EDE. An exemplary detection antibody amenable with the methods of the invention is anti-αSyn antibody clone 4B12 (BioLegend Cat. No. 807804). In another embodiment, the antibody comprises at least the antigen-binding region of 4B12. In another embodiment, the antibody comprises at least the hypervariable region (CDR sequences) of 4B12. In another embodiment, the antibody is specific to substantially the same epitope specificity as 4B12. In another embodiment, the at least one detection antibody is directed to an epitope comprising residues 103-108 on a human αSyn polypeptide. Each possibility represents a separate embodiment of the invention.

In another embodiment, the system comprises at least three populations of fluorescence-labeled magnetic microspheres. In another embodiment, each population of the distinct fluorescence-labeled magnetic microspheres comprises a distinct combination of fluorophores, enabling its discrimination from the other microspheres populations. In another embodiment said fluorescence-labeled magnetic microspheres are fluorescent magnetic microspheres compatible with Luminex detection devices (e.g. MagPlex microspheres).

In another embodiment, the targets are selected from the group consisting of GAP43, PLP-1. P2RY12 and combinations thereof. In another embodiment, the targets are GAP43, PLP-1, and P2RY12. In another embodiment, the system comprises a population of magnetic microspheres displaying an antibody directed to GAP43, labeled by a first combination of fluorophores, a second population of magnetic microspheres displaying an antibody directed to PLP-1, labeled by a second combination of fluorophores, and/or a third population of magnetic microspheres displaying an antibody directed to P2RY12, labeled by a third combination of fluorophores. Each possibility represents a separate embodiment of the invention.

In another embodiment, the method does not include additional steps of EV isolation and/or sample processing, intended to enrich the biofluid sample with EV prior to incubation with the system. For example, the methods of the invention are herein demonstrated to provide accurate diagnosis for the presence of a synucleinopathy using unprocessed plasma samples of e.g., 50 or 25 μl plasma (optionally diluted to a total volume 50 μl for the use in a Luminex-based assay assay), without employing EV immunoprecipitation, size exclusion chromatography or similar steps that were required in hitherto reported assays.

In another embodiment, the method further comprises treating the subject determined to be afflicted with a synucleinopathy with a suitable drug or treatment. For example, the subject may be treated with a drug selected from the group consisting of Levodopa, Dopamine agonists (e.g. pramipexole, ropinirole and rotigotine), Apomorphine, MAO B and COMT inhibitors, Anticholinergics and Amantadine. In another embodiment said method comprises treating said subject with a PD-specific therapy. In another embodiment, the PD-specific therapy is selected from the group consisting of dopamine precursors, dopamine agonists, and MAO-B inhibitors.

In another embodiment, the method further comprises treating the subject identified with the presence of a synucleinopathy (e.g. Lewy body dementia or multiple system atrophy) with a suitable drug or treatment. For example, the subject may be treated with a drug selected from the group consisting of cholinesterase inhibitors, and medications that increase blood pressure. In another embodiment, said subject may further be treated by and medications that manage parkinsonism symptoms (e.g. as disclosed herein).

In another embodiment, the method further comprises treating the subject identified with the presence of Lewy body dementia, to avoid administration of harmful drugs contraindicated for synucleinopathies. For example, Lewy body dementia patients should not be treated with first-generation antipsychotics (FGA) as these drugs can cause severe confusion, severe parkinsonism, sedation and sometimes death.

In another embodiment, the method further comprises treating the subject identified with PD with dementia (PDD) to improve management of symptoms (e.g. by Levmotor), as well as avoid FGA as these can increase the parkinsonism symptoms.

In another aspect, the invention relates to a method for determining the compatibility of an assay or reagent for the diagnosis of a synucleinopathy (and/or for use in a method as disclosed herein), comprising assessing the EV selectivity of the assay or reagent, wherein if said assay or reagent is determined to be capable of selectively identifying αSyn on the surface of an EV populations of a neuronal or glial origin (in particular, a GAP43, PLP-1 and/or P2RY12-displaying population), and not on an EDE population (in particular, a CD235a-displaying population), said assay or reagent is determined to be compatible with the diagnosis of a synucleinopathy (or for use in a method as disclosed herein). In another embodiment, the method further comprises determining that said assay or reagent is capable of detecting membrane-bound αSyn specifically without detecting intracellular or intra-vesicular αSyn. In another embodiment, the method is used for determining the compatibility of said assay or reagent according to a method of the invention (e.g., a method to determine the presence or absence of a synucleinopathy as disclosed herein). In another embodiment, the method further comprises determining the presence or absence of a synucleinopathy in a subject in need thereof, as disclosed herein. In another embodiment, the method further comprises treating the subject identified with the presence of a synucleinopathy with a synucleinopathy-specific drug or treatment as disclosed herein.

In another aspect, there is provided a method of evaluating a synucleinopathy, or a synucleinopathy-associated condition, the method comprising selectively assessing the level of at least one membrane-bound αSyn form, specifically on the surface of at least one neuronal or glial EV population, in a biofluid sample of the subject. In another embodiment, the method comprises analyzing EV populations in a sample of a subject, as disclosed herein.

In another aspect, the invention provides a method of analyzing extracellular vesicle (EV) populations in a sample of a subject, the method comprising:
a. providing a capture system, comprising at least three populations of distinct fluorescence-labeled magnetic microspheres, wherein each microsphere population displays antibodies directed to distinct targets on the surface of distinct neural and/or glial EV populations,
b. providing a blood-derived sample of the subject, the sample comprising less than 75 μl of non-processed plasma, or a corresponding amount of intact EV;
c. incubating the sample with the capture system, under conditions such as to allow specific antigen-antibody binding while substantially maintaining the integrity of the EV membranes, to thereby provide distinct populations of EV-microsphere complexes corresponding to each target;
d. washing the EV-microsphere complexes using a magnetic device, under conditions enabling selective capturing of said complexes;
e. incubating the captured complexes with at least one labeled detection antibody, the antibody directed to a neuronal or glial membrane-bound α-synuclein, under conditions such as to allow specific antigen-antibody binding while substantially maintaining the integrity of the EV membranes;
f. washing the resulting labeled complexes using a magnetic device to remove excess reagents;
g. subjecting the resulting complexes to a microfluidic device amenable for simultaneously detecting and quantifying fluorescent emission on a plurality of wave lengths, to thereby quantify the fluorescence emission levels and provide a separate assessment of the α-synuclein level corresponding to each of the EV populations; and
h. comparing the assessed levels to control levels;
   wherein the method is performed using reagents and under conditions so as to retain said EV in a substantially intact form.

In yet another aspect, there is provided a kit for evaluating or diagnosing a synucleinopathy, comprising:
i) a capture system, comprising a first population of magnetic microspheres displaying an antibody directed to GAP43, and labeled by a first combination of fluorophores, a second population of magnetic microspheres displaying an antibody directed to PLP-1, and labeled by a second combination of fluorophores, and a third population of magnetic microspheres displaying an antibody directed to P2RY12, and labeled by a third combination of fluorophores;
ii) at least one detection antibody capable of selectively identifying αSyn on the surface of an EV populations of a neuronal or glial origin, and not on an EDE population; and optionally
iii) reagents for performing said evaluation under conditions so as to retain said EV in a substantially intact form.

Other objects, features and advantages of the present invention will become clear from the following description and drawings.

### BRIEF DESCRIPTION OF THE DRAWINGS

FIG. 1 depicts detection of αSyn on EV captured with anti-GAP43 beads in an intact exosome Luminex assay, using the following detection antibodies: anti-αSyn antibodies clones 4B12; Syn211 (“S211”); MJFR1 (“MJFR”); and BD Biosciences clone 42 (clone 42) or a pan-tetraspanin antibody cocktail used as positive control (“pTSPN”).

FIG. 2A-2C depict αSyn detection on the surface of EV from different cellular sources using the different anti-αSyn antibodies, in healthy control subjects (“control”, open circles) and Parkinson's disease patients (“PD”, filled circles). FIG. 2A—αSyn detection on neuronal, microglial and oligodendrocyte (ODN)-derived EV from PD patients and controls, using the 4B12 detection antibody. FIG. 2B—detection of αSyn on erythrocyte, neuronal, microglial and ODN-derived EV from PD patients and controls using the Syn211 detection antibody. FIG. 2C—detection of phosphorylated αSyn on erythrocyte, neuronal, microglial and ODN-derived EV from PD patients and controls using the S129P detection antibody.

FIG. 3A-3C illustrate the level of correlation between membrane-bound αSyn measurements in distinct EV populations for each test subject, using 25 μl plasma sample input for each measurement. Individual subjects are shown. Circles mark individuals in which the αSyn levels in EV of a particular cell of origin are significantly higher compared to other EV populations. FIG. 3A—αSyn measurements in neuron (Y axis) and oligodendrocyte-derived EVs (X axis). FIG. 3B—αSyn measurements in microglia (Y axis) and oligodendrocyte-derived EVs (X axis). FIG. 3C—measurement of αSyn levels in microglia (Y axis) and neuron-derived EV (X axis).

FIG. 4A-4H illustrate the fluorescent signal measured from EV detected with either anti-αSyn or anti-pSyn antibodies, following capture by anti-GAP43 or PLP1 antibodies, as a function of sample volume (FIGS. 4A-4B and 4E-4F) or assay parameters (FIGS. 4C-4D and 4G-4H). The assay was performed in four technical replicates for each detection antibody, using decreasing volumes of samples obtained from two PD patients (“PD1” and “PD2”) and one healthy control individual (“Control”). FIG. 4A—signal obtained with anti-GAP43 capture antibody and 4B12 detection antibody, using input plasma volumes of 50, 25, 12.5, 6.25, 3.125 and 1.56 μl; FIG. 4B—signal obtained with anti PLP1 capture antibody and 4B12 detection antibody, using the different plasma sample volumes; FIG. 4C—signal obtained with anti-GAP43 capture antibody and 4B12 detection antibody, on intact EV (“untreated”, black bars) or EV treated by 1% Triton-X100 (“TX-100”, white bars) or excess soluble recombinant αSyn protein (“rα-Syn”, hatched bars); FIG. 4D—signal obtained with anti PLP1 capture antibody and 4B12 detection antibody on untreated, TX-100-treated, or rα-Syn-treated samples; FIG. 4E—signal obtained with anti-GAP43 capture antibody and anti-pSyn detection antibody, using indicated input plasma volumes; FIG. 4F—signal obtained with anti PLP1 capture antibody and anti-pSyn detection antibody, using the indicated input plasma volumes; FIG. 4G—signal obtained with anti-GAP43 capture antibody and anti-pSyn detection antibody, using untreated, TX-100-treated, or rα-Syn-treated samples; FIG. 4H—signal obtained with anti PLP1 capture antibody and anti-pSyn detection antibody, on untreated, TX-100-treated, or rα-Syn-treated samples.

FIG. 5A-5D compares the results of two independent analyses of the assays illustrated in FIG. 2, with the same PD and control plasma samples, denoted by either “PD” or “C”, respectively. FIG. 5A-5B—detection of αSyn (with anti-αSyn clone 4B12 detection antibody) and phosphorylated αSyn (pSyn, with anti-pSyn S129P detection antibody), respectively, on the surface of EV captured with either anti-GAP43, P2RY12 or PLP1 capture antibodies, as in the experiment described in FIG. 2 (Experiment 1). FIG. 5C-5D—detection of αSyn and pSyn, respectively, on the surface of EV captured either anti-GAP43, P2RY12 or PLP1 capture antibodies, in a second, independent measurement (Experiment 2).

FIG. 6A-6F show the correlation between the results of the two repeat experiments presented in FIG. 5 (experiments 1 and 2 are plotted on axes X and Y, respectively), along with a statistical analysis (Pearson correlation coefficients). FIGS. 6A and 6B demonstrate the detection αSyn and phosphorylated αSyn (pSyn), respectively, on the surface of EV captured with anti-GAP43 antibody. FIGS. 6C and 6D demonstrate the detection αSyn and pSyn (respectively) on the surface of EV captured with anti-P2RY12 antibody. FIGS. 6E and 6F demonstrate the detection αSyn and pSyn (respectively) on the surface of EV captured with anti-PLP1 antibody.

FIG. 7A-7C show measurements of surface-bound αSyn levels in EVs from plasma of subjects with Parkinson's Disease (PD), Lewy Body Disease (LBD) and a sub-population of Alzheimer's Disease (AD) characterized by mixed pathologies. FIG. 7A shows the results for plasma samples of 32 PD patients and 17 age matched healthy controls. FIG. 7B shows the results for plasma samples of 12 healthy controls, 11 PD patients, 18 LBD patients, and 11 AD patients. FIG. 7C shows an ROC curve generated from the measurements of PD and healthy controls from both cohorts (A and B), demonstrating the potential of the method to identify PD with 84% sensitivity and 78% specificity.

### DETAILED DESCRIPTION OF THE INVENTION

The present invention relates to improved and minimally invasive biomarker-based diagnostics for synucleinopathies, including for example Parkinson's disease (PD), Lewy body dementia (LBD), PD with dementia (PDD), pure autonomic failure (PAF), multiple system atrophy (MSA) and mixed Alzheimer's disease (AD) pathology. The invention further provides assays and methods for analyzing biological samples for the evaluation and determination of characteristics pertaining to synucleinopathies, and to methods for determining the compatibility of analytical agents with diagnosis of a synucleinopathy (e.g., PD). More specifically, the invention in embodiments thereof relates to improved methods comprising quantification of α-synuclein-based biomarkers on the surface of extracellular vesicles (EV).

The invention is based, in part, on the development of an unexpectedly improved assay for detecting and analyzing EV-associated biomarkers, providing for specific quantification of α-synuclein (αSyn) forms in low volume plasma samples. In particular, demonstrated herein is the successful capture of intact EV of four distinct cellular origins, and simultaneous detection and quantification of surface-bound αSyn on each EV population using a Luminex-based assay. Notably, the assay was even capable of identifying and quantifying phosphorylated α-synuclein (p-αSyn), which is typically present at significantly lower levels on the surface of EV, thus requiring a large initial sample volume in order to meet the threshold for detection.

The invention is further based, in part, on the surprising discovery, that αSyn-specific antibodies differ in their tissue selectivity and in their ability to differentiate patients with synucleinopathies from healthy controls. Specifically, using the assays developed and disclosed herein, the Syn211 anti-αSyn antibody was found to be capable of detecting αSyn on neural-derived EV (NDE), as well as on oligodendrocyte, microglia and erythrocyte-derived EV, but was not able to differentiate PD patients from healthy subjects; in contradistinction, the 4B12 anti-αSyn antibody, which did not identify αSyn on erythrocyte-derived EV, was able to differentiate the PD and control groups with high accuracy. In other words, the ability to detect αSyn on erythrocyte EV was unexpectedly found to be correlated with poor diagnostic capacity, whereas selectivity towards αSyn forms presented on EV from neural cell populations was correlated with enhanced diagnostic capacity.

Accordingly, using the improved methodology disclosed herein and employing the unexpected findings relating to antibody specificity, diagnostic assays for synucleinopathies (e.g., PD) were developed, and determined to provide unexpectedly high accuracy in differentiating patients with synucleinopathies from healthy controls.

In some embodiments, there is provided herein a method for assessing or determining the presence or absence of a synucleinopathy in a subject. In additional embodiments, there is provided herein a method for assessing or determining the presence or absence of a synuclein pathology in a subject. In additional embodiments, there is provided herein a method for aiding the diagnosis of synucleinopathies, including Parkinson's disease (PD), Lewy body dementia (LBD), PD with dementia (PDD), pure autonomic failure (PAF), multiple system atrophy (MSA) and mixed Alzheimer's disease (AD) pathology.

The methods disclosed herein may be combined with additional clinical parameters/clinical manifestations/tests to determine the type of the synucleinopathy (e.g., Parkinson's disease, Lewy body dementia, etc.).

According to a first aspect of the invention, there is provided a method of determining the presence or absence of PD in a subject in need thereof, comprising selectively assessing the level of at least one membrane-bound αSyn form, specifically on the surface of at least one neuronal or glial EV population, in a biofluid sample of the subject.

In one embodiment, the selective assessment is performed using a reagent that specifically binds αSyn on neuronal and glial EV, and does not specifically bind αSyn on erythrocyte-derived EV. In another embodiment, the method comprises selectively assessing the levels of membrane-bound αSyn specifically on the surface of neural-derived EV (NDE), oligodendrocyte-derived EV (ODE), and microglia-derived EV (MDE). In another embodiment, said biofluid sample is a blood-derived sample comprising less than 75 μl of non-processed plasma, or a corresponding amount of intact EV. In another embodiment, said sample comprises 1-50 μl of plasma or serum, and the levels of membrane-bound αSyn on the surface of NDE, ODE, and MDE are assessed simultaneously from said sample.

In another embodiment, the method comprises the steps of:
a. providing a capture system, comprising at least three populations of distinct fluorescence-labeled magnetic microspheres, wherein each microsphere population displays antibodies directed to distinct targets on the surface of distinct neural and/or glial EV populations,
b. providing a blood-derived sample of the subject, the sample comprising less than 75 μl of non-processed plasma, or a corresponding amount of intact EV;
c. incubating the sample with the capture system, under conditions such as to allow specific antigen-antibody binding while substantially maintaining the integrity of the EV membranes, to thereby provide distinct populations of EV-microsphere complexes corresponding to each target;
d. washing the EV-microsphere complexes using a magnetic device, under conditions enabling selective capturing of said complexes;
e. incubating the captured complexes with at least one labeled detection antibody, the antibody directed to a neuronal or glial membrane-bound αSyn, under conditions such as to allow specific antigen-antibody binding while substantially maintaining the integrity of the EV membranes;
f. washing the resulting labeled complexes using a magnetic device to remove excess reagents;
g. subjecting the resulting complexes to a microfluidic device amenable for detecting and simultaneously quantifying fluorescent emission on a plurality of wave lengths, to thereby quantify the fluorescence emission levels and provide a separate assessment of the αSyn level corresponding to each of the EV populations (for each of the labeled detection antibodies); and
h. comparing the assessed levels to control levels;
   wherein the method is performed using reagents and under conditions so as to retain said EV in a substantially intact form.

In another embodiment, the at least one membrane-bound α-synuclein form is detected by at least one distinct-fluorescently labeled detection antibody selected from the group consisting of: an antibody directed to non-phosphorylated αSyn, an antibody directed to phosphorylated αSyn, and an antibody directed to both phosphorylated and non-phosphorylated αSyn. In another embodiment, the at least one detection antibody is directed to an epitope comprising residues 103-108 on a human αSyn polypeptide.

In another embodiment, the assessed αSyn levels represent normalized levels. For example, the αSyn level corresponding to each of the EV populations (as assessed for each of the labeled detection antibodies) may be further normalized to the total amount of EV corresponding the respective EV population, and, for instance, the αSyn level assessed in NDE may be normalized to the assessed amount of NDE assessed in the sample, the αSyn level assessed in EDE may be normalized to the amount of EDE assessed in the sample, and the αSyn level assessed in MDE may be normalized to the amount of MDE assessed in the sample. In some embodiments, assessment of the total (or relative) amounts of each EV population may conveniently be done using positive control surface markers characteristic of EV (either tissue-specific or non-tissue-specific, the latter also referred to herein as a general EV marker). According to some embodiments, the assessment of the amount of the EV populations may be performed by systems and methods as described herein, in which the detection antibody used in step e. is replaced by at least one labeled detection antibody directed to a general EV marker (e.g. a tetraspanin marker including, but not limited to CD63 and CD81). Conveniently, when normalized αSyn levels are used, the control levels of step h. also represent normalized control levels, in which αSyn level measured e.g. on NDE of a sample of a healthy control subject is also assessed and normalized to total NDE as done in the sample of the test subject (provided in step b.).

In another embodiment, a level of the at least one membrane-bound αSyn form that is significantly higher than the level corresponding to a healthy control subject, indicates the presence of a synucleinopathy (e.g., PD) in said subject. In another embodiment a level of the at least one membrane-bound αSyn form that is not substantially higher than the level corresponding to a healthy control subject, indicates the absence of synucleinopathy (e.g., PD) in said subject. In another embodiment, the method comprises comparing the levels of the at least one membrane-bound αSyn form as assessed in each of said EV populations to their respective levels corresponding to a control sample, to thereby compare the diagnostic signature of the sample to the control diagnostic signature, wherein a significant difference in the diagnostic signature of the subject compared to the control diagnostic signature indicates that said subject is afflicted with synucleinopathy (e.g., PD).

In another embodiment, the targets are selected from the group consisting of GAP43, PLP-1, P2RY12 and combinations thereof. In another embodiment, the system comprises a first population of magnetic microspheres displaying an antibody directed to GAP43, and labeled by a first combination of fluorophores, a second population of magnetic microspheres displaying an antibody directed to PLP-1, and labeled by a second combination of fluorophores, and a third population of magnetic microspheres displaying an antibody directed to P2RY12, and labeled by a third combination of fluorophores.

In another embodiment, the method further comprises treating the subject determined to be afflicted with PD with a PD-specific therapy. In another embodiment, the PD-specific therapy is selected from the group consisting of dopamine precursors, dopamine agonists, and MAO-B inhibitors. In another embodiment, the PD-specific therapy is selected from the group consisting of dopamine precursors, dopamine agonists, NDMA receptor antagonists and MAO-B inhibitors.

In another aspect, there is provided a method for analyzing EV populations in a sample of a subject, the method comprising:
a. providing a capture system, comprising at least three populations of distinct fluorescence-labeled magnetic microspheres, wherein each microsphere population displays antibodies directed to distinct targets on the surface of distinct neural and/or glial EV populations,
b. providing a blood-derived sample of the subject, the sample comprising less than 75 μl of non-processed plasma, or a corresponding amount of intact EV;
c. incubating the sample with the capture system, under conditions such as to allow specific antigen-antibody binding while substantially maintaining the integrity of the EV membranes, to thereby provide distinct populations of EV-microsphere complexes corresponding to each target;
d. washing the EV-microsphere complexes using a magnetic device, under conditions enabling selective capturing of said complexes;
e. incubating the captured complexes with at least one labeled detection antibody, the antibody directed to a neuronal or glial membrane-bound α-synuclein, under conditions such as to allow specific antigen-antibody binding while substantially maintaining the integrity of the EV membranes;
f. washing the resulting labeled complexes using a magnetic device to remove excess reagents;
g. subjecting the resulting complexes to a microfluidic device amenable for detecting and simultaneously quantifying fluorescent emission on a plurality of wave lengths, to thereby quantify the fluorescence emission levels and provide a separate assessment of the α-synuclein level corresponding to each of the EV populations; and
h. comparing the assessed levels to control levels;
wherein the method is performed using reagents and under conditions so as to retain said EV in a substantially intact form.

In another embodiment, the sample is a plasma or serum sample. In another embodiment, said sample comprises 1-50 μl of plasma or serum. In another embodiment, said sample is obtained from a subject afflicted with, or suspected of having, a synucleinopathy, or a synucleinopathy-associated condition. In another embodiment, the synucleinopathy is associated with a condition selected from the group consisting of Parkinson's disease (PD), Lewy body dementia (LBD), PD with dementia (PDD), pure autonomic failure (PAF), and multiple system atrophy (MSA). In another embodiment, the method further comprises diagnosing or evaluating a condition selected from the group consisting of PD, LBD, PDD, PAF, and MSA, in said subject. Each possibility represents a separate embodiment of the invention.

In another embodiment, said subject is suspected of having PD. In another embodiment, αSyn levels that are significantly higher than the levels corresponding to a healthy control subject, indicate the presence of PD in said subject, and/or wherein an αSyn levels that are not substantially higher than the levels corresponding to a healthy control subject, indicate the absence of PD in said subject.

In another embodiment, the subject is diagnosed with, or is suspected of having, a dementia or cognitive decline. In another embodiment, αSyn levels that are significantly higher than the levels corresponding to a healthy control subject indicate the presence of a dementia or cognitive decline associated with αSyn pathology. In a particular embodiment, said subject is diagnosed with, or is suspected of having, a dementia or cognitive decline associated with LBD. In another particular embodiment, said subject is diagnosed with, or is suspected of having, a dementia or cognitive decline associated with PDD. Each possibility represents a separate embodiment of the invention.

In another embodiment, the method further comprises determining treatment for said subject. In another embodiment, determining treatment comprises determining that said subject in amenable for treatment with one or more agents indicated for management of a synucleinopathy or a condition associated therewith. In another embodiment, determining treatment comprises determining that said subject in not amenable for treatment with one or more agents that are contraindicated for, or excluded from management of, a synucleinopathy or a condition associated therewith.

In another embodiment, the subject is determined to be afflicted with a dementia or cognitive decline associated with αSyn pathology, and the method comprises that said subject is not amenable for treatment with one or more agents selected from the group consisting of: anticholinergic drugs, dopamine precursors, dopamine agonists, and first-generation antipsychotics (FGA). In a particular embodiment, said dementia or cognitive decline is associated with LBD. In another particular embodiment, said dementia or cognitive decline is associated with PDD. Each possibility represents a separate embodiment of the invention.

In an additional embodiment, the method further comprises treating the subject determined to be afflicted with the synucleinopathy or synucleinopathy-associated condition, with one or more agents indicated for management of said synucleinopathy or a condition associated therewith.

In an additional embodiment, the method further comprises selecting at least one labeled detection antibody to be used in step c. as an antibody capable of selectively identifying αSyn on the surface of an EV populations of a neuronal or glial origin, and not on an EDE population. In a particular embodiment, said antibody is capable of selectively identifying αSyn on the surface of an GAP43, PLP-1and/or P2RY12-displaying EV populations, and not on a CD235a-displaying EV population.

In another aspect, the invention provides a method for determining the compatibility of an assay or reagent for the diagnosis of synucleinopathy (e.g., PD), comprising assessing the EV selectivity of the assay or reagent, wherein if said assay or reagent is determined to be capable of selectively identifying αSyn on the surface of an EV populations of a neuronal or glial origin, and not on an EDE population, said assay or reagent is determined to be compatible with the diagnosis of synucleinopathy (e.g., PD). In another embodiment, said assay or reagent comprises an antibody. In another embodiment, said antibody is capable of selectively identifying αSyn on the surface of an GAP43, PLP-1 and/or P2RY12-displaying EV populations, and not on a CD235a-displaying EV population.

In another aspect, there is provided a kit for evaluating or diagnosing a synucleinopathy, comprising:
i) a capture system, comprising a first population of magnetic microspheres displaying an antibody directed to GAP43, and labeled by a first combination of fluorophores, a second population of magnetic microspheres displaying an antibody directed to PLP-1, and labeled by a second combination of fluorophores, and a third population of magnetic microspheres displaying an antibody directed to P2RY12, and labeled by a third combination of fluorophores;
ii) at least one detection antibody capable of selectively identifying αSyn on the surface of an EV populations of a neuronal or glial origin, and not on an EDE population; and optionally
iii) reagents for performing said evaluation under conditions so as to retain said EV in a substantially intact form.

In another embodiment, the magnetic microspheres are further coated with negatively-charged peptides amenable for diminishing non-specific interactions. In another embodiment, at least one detection antibody is fluorescently labeled and is capable of selectively identifying αSyn on the surface of an GAP43, PLP-1 and/or P2RY12-displaying EV populations, and not on a CD235a-displaying EV population. In an additional embodiment, the at least one detection antibody is fluorescently labeled by quantum dots or by combinations of multiple fluorophores.

In another embodiment, the reagents are selected from the group consisting of:
(i) at least one binding buffer for incubating a sample with the capture system to thereby provide distinct populations of EV-microsphere complexes, the at least one binding buffer characterized by lack of detergents and by the presence of protease and/or phosphatase inhibitors;
(ii) at least one washing buffer, characterized by significantly enhanced salt concentrations compared to the at least one binding buffer; and
(iii) at least one binding buffer and at least one washing buffer as defined in (i) and (ii) above.

These and other embodiments are described in further detail below.

### Synucleinopathies

Alpha synuclein (αSyn), while widely accepted as a major pathophysiological driver of Parkinson's disease (PD), is becoming increasingly implicated in other neurodegenerative diseases, collectively termed “synucleinopathies”. αSyn is a 14 kDa protein expressed abundantly in the in the presynaptic terminals of the brain, where, under abnormal circumstances, it forms neurotoxic aggregates that have detrimental effects on neuronal activity. When aberrant accumulation of αSyn is known to be the main pathological contributor, the neurodegenerative situation is referred to as a “primary synucleinopathy”, examples include Lewy body disorders such as Lewy body dementia. PD with dementia (PDD) and pure autonomic failure (PAF). A further synucleinopathy, multiple system atrophy (MSA) is characterized by glial cytoplasmic inclusions (Papp-Lantos bodies). All synucleinopathies are considered idiopathic diseases, meaning their etiology is currently unknown. Abnormal accretion of αSyn is frequently observed in brains with aberrant deposition of Tau, transactive response DNA binding protein 43 kDa (TDP-43), amyloid-β (Aβ) or prion protein. Indeed, the co-occurrence of these anomalous protein aggregations is so widespread, they may be considered a typical feature of most neurodegenerative pathologies. While it is not clear yet if patients suffer from multiple diseases, synuclein related and unrelated, or from mixed pathologies that are all part of the same disease, it is clear that detailed characterization of the pathology, including α-synuclein level is needed to achieve accurate diagnosis and precision medicine.

While synucleinopathies vary in prevalence, symptom patterns, and severity among disorders, they all have in common autonomic nervous system dysfunctions. Typical autonomic symptoms can appear years before the indicative motor symptoms, and include constipation, urinary and sexual dysfunction, and cardiovascular autonomic symptoms such as orthostatic hypotension, supine hypertension, and reduced heart rate variability. Seeing as how there are currently no biological assays or biomarker measurements of any kind allowing for the definitive diagnosis of any synucleinopathy, the diagnosis is based upon said symptoms and additional clinical attributes of the patient. The onset of Parkinsonism, a term denoting the syndrome of motor-related symptoms, is usually the time point of initial clinical intervention, at which stage the patient is unfortunately on a clear path of deterioration with little to no prognostic hope. The progression of the disease elucidates additional symptoms by which a differential diagnosis is possible, allowing for a more informed speculation as to the nature of the specific synucleinopathy.

Due to the overlapping symptoms of the different synucleinopathies, complicated by a lack of unequivocal diagnostic measures, existing pharmaceutical interventions are solely aimed at symptom management.

A candidate for PD diagnosis (which may also be referred to in embodiments of the invention as a subject suspected of having PD), presents with characteristic unilateral resting tremor, decreased movement, or rigidity. Additional symptoms include as infrequent blinking, lack of facial expression, and gait abnormalities. Postmortem evaluation confirms the presence of synuclein-filled Lewy bodies in the nigrostriatal system and consequent degradation of dopaminergic neurons. Accordingly, the main PD-targeted pharmacology consists of dopamine-increasing agents such as Levodopa, which is the metabolic precursor of dopamine, crossing the blood-brain barrier into the basal ganglia, where it is decarboxylated to form dopamine. Amantadine, an NMDA-receptor antagonist, is useful as monotherapy for early, mild parkinsonism and later can be used to augment levodopa's effects, as well as anticholinergic drugs. Another class of frequently prescribed medications for PD are dopamine agonists, which directly activate dopamine receptors in the basal ganglia, examples include Pramipexole, Ropinirole, Rotigotine and Apomorphine. An additional pharmacological approach pertains to the inhibition of dopamine degrading enzymes in the brain via MAO-B inhibitors. It is interesting to note that responsiveness to levodopa is used by clinicians to help distinguish PD from secondary or atypical parkinsonism. Of the different synucleinopathies, PD is the only one with pharmacological agents with a specific indication for the disease.

Lewy bodies, the cytoplasmic inclusions of αSyn aggregates, occur not only in PD but also in two highly overlapping types of dementia—PD dementia (PDD) and Lewy Body Dementia (LBD), both of which are progressive neuronal degradations presenting as deteriorating cognitive skills with a poor prognosis. LBD manifests with early and prominent deficits in attention, executive function, and visuoperceptual ability. LBD's Extrapyramidal symptoms begin within one year of the cognitive symptoms, unlike in PD. Also, the extrapyramidal symptoms differ from those of Parkinson disease; in dementia with Lewy bodies, tremor does not occur early, rigidity of axial muscles with gait instability occurs early, and deficits tend to be symmetric. The main therapeutic avenue is amelioration of symptom severity, main pharmacological agents being cholinesterase inhibitors which improve cognition.

MSA is a differential diagnosis of PD, based upon unresponsiveness to Levodopa. It is a synucleinopathy which presents as a combination of Parkinsonism, cerebellar abnormalities and manifestations of autonomic insufficiency. Supportive care targets the assorted symptoms like Orthostatic hypotension, incontinence, constipation and erectile dysfunction, with standard and appropriate clinical practices.

In some embodiments, a subject may be determined to be suspected of having a synucleinopathy (e.g. PD) based on the existence of symptoms or clinical presentation as disclosed herein. In other embodiments, a subject may be suspected of having (or being predisposed to developing) said synucleinopathy (e.g. PD) based on the existence of other familial, genetic or environmental factors known in the art.

### Antibodies

The invention in embodiments thereof relates to the use of binding reagents, including in particular antibodies. As used herein in the context of embodiments of the invention, the term antibody relates to at least an antigen-binding portion of an antibody.

An antibody directed (or specific) to an antigen, as used herein is an antibody which is capable of specifically binding the antigen. The term “specifically bind” or “specifically recognize” as used herein means that the binding of an antibody to an antigen is not competitively inhibited by the presence of non-related molecules.

Intact antibodies include, for example, polyclonal antibodies and monoclonal antibodies (mAbs). Exemplary functional antibody fragments comprising whole or essentially whole variable regions of both light and heavy chains (forming an antigen-binding portion) include, for example: (i) Fv, defined as a genetically engineered fragment consisting of the variable region of the light chain and the variable region of the heavy chain expressed as two chains; (ii) single-chain Fv (“scFv”), a genetically engineered single-chain molecule including the variable region of the light chain and the variable region of the heavy chain, linked by a suitable polypeptide linker; (iii) Fab, a fragment of an antibody molecule containing a monovalent antigen-binding portion of an antibody molecule, obtained by treating whole antibody with the enzyme papain to yield the intact light chain and the Fd fragment of the heavy chain, which consists of the variable and CH1 domains thereof; (iv) Fab′, a fragment of an antibody molecule containing a monovalent antigen-binding portion of an antibody molecule, obtained by treating whole antibody with the enzyme pepsin, followed by reduction (two Fab′ fragments are obtained per antibody molecule); and (v) F(ab′)2, a fragment of an antibody molecule containing a monovalent antigen-binding portion of an antibody molecule, obtained by treating whole antibody with the enzyme pepsin (i.e., a dimer of Fab′ fragments held together by two disulfide bonds). Further included within the scope of the invention are chimeric antibodies; recombinant and engineered antibodies, single-chained antibodies (e.g. single-chain Fv) and fragments thereof (comprises the antigen-binding portion). The term “antigen” as used herein is a molecule or a portion of a molecule capable of being bound by an antibody. The antigen is typically capable of inducing an animal to produce antibody capable of binding to an epitope of that antigen. An antigen may have one or more epitopes. The specific reaction referred to above is meant to indicate that the antigen will react, in a highly selective manner, with its corresponding antibody and not with the multitude of other antibodies which may be evoked by other antigens.

Methods of generating monoclonal and polyclonal antibodies are well known in the art. Antibodies may be generated via any one of several known methods, which may employ induction of in vivo production of antibody molecules, screening of immunoglobulin libraries, or generation of monoclonal antibody molecules by continuous cell lines in culture. These include, but are not limited to, the hybridoma technique, the human B-cell hybridoma technique, and the Epstein-Barr virus (EBV)-hybridoma technique. Besides the conventional method of raising antibodies in vivo, antibodies can be generated in vitro using phage display technology, by methods well known in the art (e.g. Current Protocols in Immunology, Colligan et al (Eds.), John Wiley & Sons, Inc. (1992-2000), Chapter 17, Section 17.1).

The variable domains of each pair of light and heavy chains form the antigen binding site. The domains on the light and heavy chains have the same general structure and each domain comprises four framework regions, whose sequences are relatively conserved, joined by three hypervariable domains known as complementarity determining regions (CDR₁₋₃). These hypervariable domains contribute to the specificity and affinity of the antigen binding site.

According to some embodiments, the antibody to be used in assays and methods of the invention comprises at least the complementarity determining region (CDR) sequences of a monoclonal antibody described herein, e.g. 4B12. Isolated complementarity-determining region peptides can be obtained by constructing genes encoding the CDR of an antibody of interest. Such genes may be prepared, for example, by RT-PCR of the mRNA of an antibody-producing cell. Ample guidance for practicing such methods is provided in the literature of the art.

### Data Analysis

According to embodiments of the invention, substantial difference or similarity of diagnostic signatures are determined considering the collective levels of the biomarkers (e.g. the level of a particular αSyn form in a particular EV population) of the signature. In some embodiments, a substantially different diagnostic signature compared to a control comprises significantly enhanced levels of a set of biomarkers as disclosed herein compared to their respective control levels. In other embodiments a substantially different diagnostic signature compared to a control comprises significantly reduced levels of a set of biomarkers as disclosed herein compared to their respective control levels. In yet other embodiments, a substantially different diagnostic signature compared to a control comprises both significantly enhanced levels of one or more markers as disclosed herein and significantly reduced levels of one or more additional markers as disclosed herein compared to their respective control levels. Each possibility represents a separate embodiment of the invention.

Advantageously, the methods of the invention can employ the use of learning and pattern recognition analyzers, clustering algorithms and the like, in order to discriminate between the diagnostic signature of a sample or subject and control diagnostic signatures as disclosed herein. For example, the methods can comprise determining the levels of biomarkers as disclosed herein in EV isolated from a biofluid sample, and comparing the resulting diagnostic signature to a control diagnostic signature using such algorithms and/or analyzers.

In certain embodiments, one or more algorithms or computer programs may be used for comparing the amount of each gene product quantified in the sample against a predetermined cutoff (or against a number of predetermined cutoffs). Alternatively, one or more instructions for manually performing the necessary steps by a human can be provided.

In some embodiments, receiver operating characteristics (ROC) analysis and AUC plus probabilistic metrics (e.g., log-loss) may be used in connection with the methods of the invention. Hypothesis-based signature development, where pre-knowledge on the biomarker role in the disease may be taken under consideration. In other embodiments, linear mixed-effect algorithms may be used to model differences in selected biomarker(s). In other embodiments, machine learning (ML) is used to evaluate the biomarkers as potential indicators of progression, exploiting temporal heterogeneous effects, as well as sparse and varying-length patient characteristics commonly seen with disease progression. Mixed-effect machine learning and long- and short-term memory (LSTM) neural networks may be used to predict changes in biomarker trajectories and to classify patients. Multivariate methods (e.g., logistic regression, K-nearest neighbor, support vector machine, and machine learning) can also be used. A class of non-linear algorithms that show better performance in small and medium-sized datasets including decision tree-based methods (i.e., random forest, gradient boosting) and support vector machines may also be used.

Algorithms for determining and comparing diagnostic signatures further include, but are not limited to, supervised classification algorithms including, but not limited to, gradient boosted trees, random forest, regularized regression, multiple linear regression (MLR), principal component regression (PCR), partial least squares (PLS), discriminant function analysis (DFA) including linear discriminant analysis (LDA), nearest neighbor, artificial neural networks, multi-layer perceptrons (MLP), generalized regression neural network (GRNN), and combinations thereof, or non-supervised clustering algorithms, including, but not limited to, K-means, spectral clustering, hierarchical clustering, gaussian mixture models, and combinations thereof.

Many of the algorithms are neural network-based algorithms. A neural network has an input layer, processing layers and an output layer. The information in a neural network is distributed throughout the processing layers. The processing layers are made up of nodes that simulate the neurons by the interconnection to their nodes. Similar to statistical analysis revealing underlying patterns in a collection of data, neural networks locate consistent patterns in a collection of data, based on predetermined criteria.

In other embodiments, principal component analysis is used. Principal component analysis (PCA) involves a mathematical technique that transforms a number of correlated variables into a smaller number of uncorrelated variables. The smaller number of uncorrelated variables is known as principal components. The first principal component or eigenvector accounts for as much of the variability in the data as possible, and each succeeding component accounts for as much of the remaining variability as possible. The main objective of PCA is to reduce the dimensionality of the data set and to identify new underlying variables.

In another embodiment, the algorithm is a classifier. One type of classifier is created by “training” the algorithm with data from the training set and whose performance is evaluated with the test set data. Examples of classifiers are discriminant analysis, decision tree analysis, receiver operator curves or split and score analysis.

The following examples are presented in order to more fully illustrate some embodiments of the invention. They should, in no way be construed, however, as limiting the broad scope of the invention.

### EXAMPLES

### Example 1. EV Capture and Detection Method Employing Luminex Technology Allows Specific Detection of Alpha-Synuclein (αSyn) and Phosphorylated Alpha-Synuclein (αSyn S129P) Using Low Volume of Plasma Samples

For the capture of EV populations, antibodies against GAP43 (neuronal marker, Thermo Fisher Scientific, Cat. No. MA5-32256S), PLP-1 (oligodendrocyte marker, Thermo Fisher Scientific, Cat. No. MA190652), P2RY12 (microglia marker, BioLegend, Cat. No. 848002) and CD235a/b (erythrocyte marker, BioLegend, Cat. No. 306602) were attached to distinct color-coded magnetic microspheres, as follows.

Preparation and characterization of intact exosome Luminex (IEL) beads was carried out as follows: antibodies were conjugated to fluorescent magnetic microspheres in desired luminescence range, all functionalized with carboxyl groups (MagPlex^{R}, Luminex Corp., Cat. No. MC1XXXX-01), using 1-Ethyl-3-(3-dimethylaminopropyl) carbodiimide (EDC) chemistry (He et al. 2007, Bioconjug Chem 18: 983-988). Bead recovery/concentration after conjugation was determined in a Countess™ 3 FL Automated Cell counter (Thermo Fisher Scientific, Cat. No. A49866) using reusable chamber slides (Thermo Fisher Scientific, Cat. No. A25750).

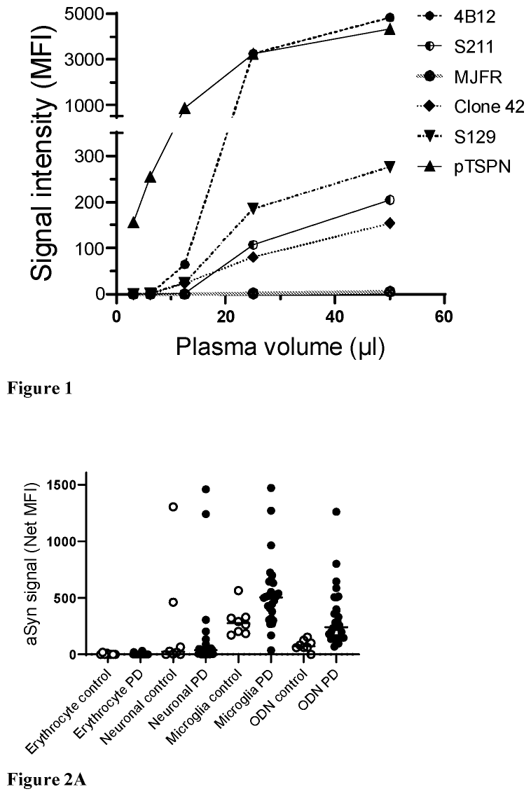

The beads were subsequently incubated with the samples, wherein different plasma volumes as indicated in FIG. 1 (also referred to herein as input volumes) were adjusted to a total assay volume of 50 μl in a detergent-free binding buffer (0.1-2% BSA in PBS, pH 7.5-8.5) containing phosphatase and protease inhibitors, and placed in duplicates in the wells of a 96 well black plate. The pull-down was carried out overnight (16-18 hrs) at 4° C. on a Genie microplate shaker (600 RPM) followed by three washes using a magnetic plate holder in a washing buffer containing increased salt concentrations (PBS with TRIS 10-100 mM, NaCl 100-500 mM, pH 7.5-8).

The resultant captured complexes were incubated with biotinylated detection antibodies against αSyn (clones 4B12, BioLegend Cat. No. 807804, “4B12”; Syn211, Thermo Fisher Scientific Cat. No. AHB0261, “S211”; MJFR1, Abcam Cat. No. ab209420, “MJFR”; and Clone 42, BD Biosciences Cat. No. 610787, “Clone 42”) or phosphorylated αSyn (S129P, BioLegend Cat. No. 825701), produced as follows: the various detection antibodies or a pan-tetraspanin antibody cocktail used as positive control (containing antibodies against CD9, CD63, and CD81; Cat. Nos. 312102, 353039 and 349502, BioLegend; “PTSPN”), were biotinylated by overnight incubation at −4° C. with EZ-link™ (Thermo Fisher Scientific Cat. No. 21442), at twenty-fold molar excess, per manufacturer's instructions. Excess biotinylation reagent was removed using Zeba™ Spin Desalting Columns, 7K MWCO (Thermo Fisher Scientific Cat. No. 89882). Incubation was performed in Assay Diluent containing biotinylated detection antibody (1-2 μg/ml, 50 μl/well) for two hours at room temperature (Genie shaker).

After another wash, streptavidin-PE (SAPE) was added and the Luminex instrument was used to gate the range for each of the antibody-conjugated microspheres, which represent exosomes derived from distinct cell types, and to measure the intensity of the corresponding PE signal. The beads were then washed three times in the washing buffer as described above, resuspended in 50 μl PBS containing Streptavidin-PE reagent (SAPE, 6 μg/ml, Biolegend Cat. No. 405204) and incubated 20 min at room temperature. Following SAPE incubation, the beads were washed three times in the washing buffer, resuspended in xMAP Sheath Fluid (Thermo Fisher Scientific Cat. No. 4050015), and the plate read on a Luminex200 reader. The Luminex instrument was used to gate the range for each of the antibody-conjugated microspheres, which represent exosomes derived from distinct cell types, and to measure the intensity of the corresponding PE signal.

The results demonstrated successful capture of intact EV of the four distinct cellular origins, and simultaneous detection and quantification of surface-bound αSyn on each EV population. Exemplary results are presented in FIG. 1, which depicts the mean fluorescent intensity (MFI) obtained with the various detection antibodies on EV captured with anti-GAP43 beads.

As can be seen in FIG. 1, all detection antibodies yielded dose dependent signals, with the exception of the MJFR1 antibody. A particularly strong fluorescent signal, comparable to that of the positive control antibody cocktail, was obtained with the 4B12 antibody.

Accordingly, the results demonstrate identification of surface-bound αSyn, with high sensitivity and reproducibility, on tissue-specific EV captured from remarkably low-volume samples. Notably, the assay was even capable of identifying and quantifying phosphorylated αSyn, which is typically present at significantly lower levels on the surface of EV, thus requiring a large initial sample volume in order to meet the threshold for detection.

### Example 2 αSyn-Specific Antibodies Differ in Their Tissue Selectivity and in Their Ability to Differentiate PD Patients From Healthy Controls

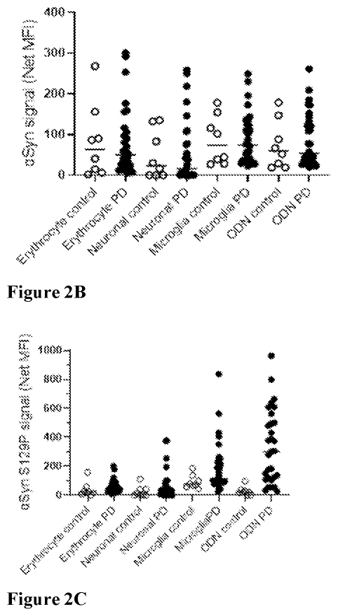

Preparation and characterization of intact exosome Luminex (IEL) beads was carried out as described in Example 1 above with the four capture antibodies (against GAP43, PLP1, P2RY12 and CD235). Three detection antibodies were selected for the second round of testing in a cohort of 8 control plasma samples and 17 Parkinson's Disease (PD) plasma samples, namely anti-αSyn antibody clones 4B12 and Syn211, and the anti-αSyn S129P antibody. Capture and detection were performed as described in Example 1. The results are presented in FIG. 2A-2C (for antibody clones 4B12, Syn211, and S129P, respectively).

Surprisingly, as can be seen in FIGS. 2A-2B, two different αSyn antibodies (clones 4B12 and Syn211) generated completely different results with respect to their ability to detect αSyn on the surface of EV from different sources. Specifically, the Syn211 antibody was able to detect αSyn on the surface of all EV populations (manifested as a modest and comparable MFI signal, FIG. 2B), while the 4B12 antibody showed a capability for selective detection of αSyn on EV captured by the GAP43, PLP, and P2RY12-specific antibodies (corresponding to neuronal, oligodendrocyte and microglial origin, respectively), but not on EV captured by the CD235-specific antibody (corresponding to erythrocytes, FIG. 2A).

Surprisingly, as can further be seen in FIG. 2B, despite the clear signal measured when the Syn211 antibody was used for detection, no significant differences were observed between EV obtained from PD patients and those obtained from healthy controls using this antibody, in any of the EV populations. In contradistinction, significant differences were observed in the EV-associated αSyn using the 4B12 antibody, in particular in EV captured by the PLP-1and P2RY12-specific antibodies (FIG. 2A). Similar results were obtained with the αSyn S129P antibody (FIG. 2C), for which differences between PD patients and controls were especially pronounced in oligodendrocytic and microglial EVs.

In summary, the assay described herein was capable of simultaneously measuring four distinct values, which represent αSyn levels associated with the particles that were captured with each of the four antibodies (corresponding to blood-borne EV derived from erythrocytes, neurons, oligodendrocytes and microglia). Further, the assay exhibited high sensitivity and accuracy, generating strong and dose dependent signals and capable of differentiating PD patients from healthy controls even when using small sample volumes.

In addition, the results demonstrate unexpected differences in the specificity of anti-αSyn antibodies for EV-bound αSyn, associated with differences in their diagnostic capacity. In particular, the ability to detect αSyn on erythrocyte EV was surprisingly found to be correlated with poor diagnostic capacity, whereas selectivity towards αSyn forms presented on EV from neural cell populations was correlated with enhanced diagnostic capacity.

### Example 3. Surface-Bound αSyn Levels in EV of Neuronal and Glial Origin

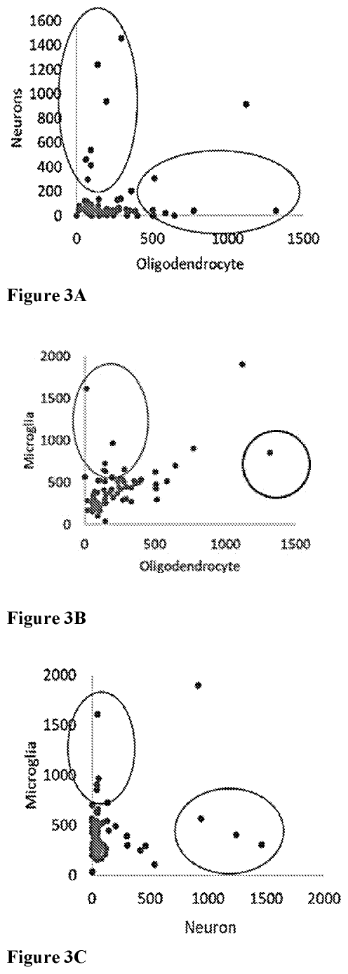

The assays described above are multiplex assays where αSyn levels on multiple cell-specific types of exosomes are measured in the same sample/well. Therefore, any experimental artifacts (besides those related to the detection antibodies) are expected to be exhibited as identical (or parallel) fluctuations in αSyn levels in NDE and other populations of EV (such as oligodendrocyte and microglial EV). FIGS. 3A-3C depict the membrane-bound αSyn level in each EV population for each test subject (including PD patients and healthy controls), as measured in samples comprising 25 μl plasma input.

However, as can be seen in FIGS. 3A-3C, no correlation was observed between neuron- microglia- or oligodendrocyte-derived exosomes with respect to surface-bound αSyn. Nor were the levels correlated with those measured on erythrocyte-derived EV. Further, the correlation between αSyn levels on microglia and oligodendrocyte exosomes was identified to be weak (FIG. 3B). In addition, the relative levels of αSyn on EV from different sources were subject-specific; for example, the ratio between αSyn associated with NDE and oligodendrocyte EV ranged between 0.1 to 10-fold among the test subjects. The circles in FIGS. 3A-3C denote individuals with much higher αSyn levels in EV of a particular cell of origin compared to the levels in EV of other cells of origin.

Thus, the results demonstrate an unexpected lack of correlation between EV from distinct cell types with respect to the relative levels of surface-bound αSyn. Accordingly, the assays disclosed herein exemplify simultaneous measurement of surface-bound αSyn in multiple non-redundant EV populations (derived from different types of cells in the nervous system), providing for improved diagnostic capacity in identifying PD patients exhibiting diverse αSyn-associated pathologies.

### Example 4. Assay Precision and Specificity

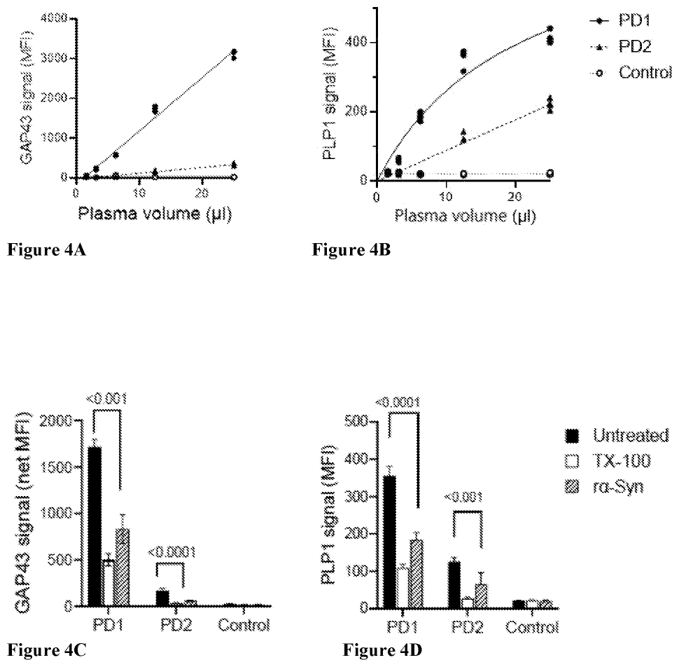

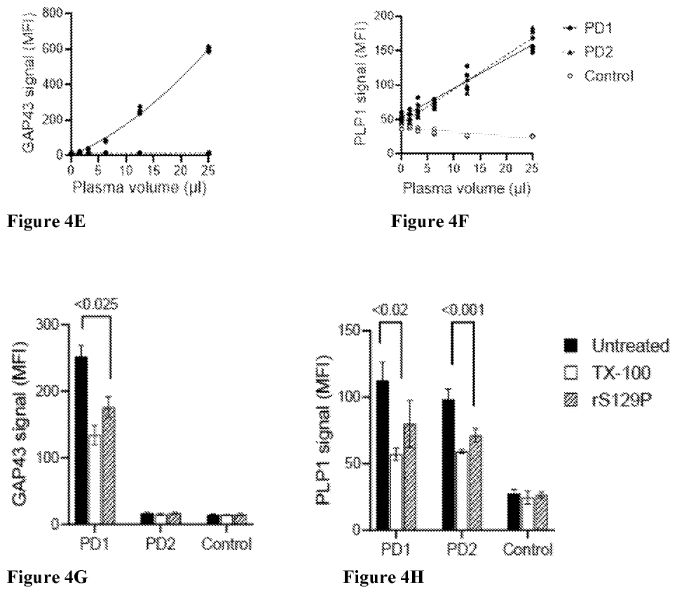

The assay was performed using decreasing sample input volumes (50, 25, 12.5, 6.25, 3.125 and 1.56 μl, adjusted to a final 50 μl assay volume with assay diluent) of three plasma samples (obtained from one control and two PD patients), each in four technical replicates. EV were captured using GAP43 and PLP-1 specific antibodies. The 4B12 antibody was used as the detection antibody in the experiments depicted in FIGS. 4A-4B, and the αSyn S129P antibody was used for detection in FIGS. 4E-4F, as described in Example 1. Each dot in FIGS. 4A-4B and 4E-4F represents a separate measurement. In addition, the lowest volume for quantification, defined as the lowest sample input volume that generates a PE signal that is two standard deviations above the blank, with a coefficient of variation (CV) below 20%, was calculated for each sample.

As can be seen in FIGS. 4A-4B, the signal (MFI) showed a linear decrease correlating with sample volume. The calculated lowest input volumes for quantification were 1.56 and 6.25 μl for the two PD samples and 12.5 μl for the control, respectively, and the CV values were below 10% for all samples when working plasma input volume was 12.5 μl or higher. Similar results were observed when the αSyn S129P antibody was used (FIGS. 4E-4F).

Thus, the results demonstrate that the assays disclosed herein provide a strong, linear fluorescent signal when quantifying surface-associated αSyn or phosphorylated αSyn on EV, captured from sample input volumes that are at least tenfold and up to ˜100-fold lower than hitherto reported assays. Accordingly, input volumes of plasma sample of 20-25 μl were determined to be advantageous and used for further analysis, as disclosed herein.

Next, the assay was performed in the presence or absence of 1% Triton-X100 (TX-100), a detergent that elicits disintegration of cellular and exosomal lipid membranes. The results are shown in FIGS. 4C-4D (for neuronal and oligodendrocyte-originated EV, respectively, using the 4B12 antibody for detection). The results show a significant reduction in MFI measured in the detergent-treated samples compared to plasma samples diluted with detergent-free buffer. Similar results were observed when the αSyn S129P antibody was used (FIGS. 4G-4H). Thus, a statistically significant reduction in the fluorescent signal for both αSyn and αSyn S129P was observed in the presence of TX-100, indicating that the signal depends on the presence of connecting lipid membranes. Accordingly, the assays as disclosed herein specifically measure αSyn and αSyn S129P on the surface of intact EV rather than in lysates of disintegrated EV.

The assay was also performed in the presence of excess soluble recombinant αSyn protein (rα-Syn) or recombinant phosphorylated protein (rS129P), in order to determine the target specificity of the assay. To this end, the detection antibodies (αSyn and αSyn S129P, respectively) were pre-incubated for 20 min with 1 μg/ml of the appropriate recombinant protein prior to being used in the assay, to block the specific interaction of the antibodies with αSyn displayed on EV surface but not the non-specific adherence. The results are shown in FIGS. 4C-4D (αSyn-specific detection antibody with neuron- and oligodendrocyte-specific capture antibodies, respectively) and 4G-4H (αSyn S129P-specific detection antibody with neuron- and oligodendrocyte-specific capture antibodies, respectively).

As can be seen in FIGS. 4C-4D and 4G-4H, incubation with soluble αSyn and αSyn S129P significantly reduced the signal. In summary, the results in FIGS. 4A-4H demonstrate the specificity of the assay to membrane-bound αSyn forms on intact EV.

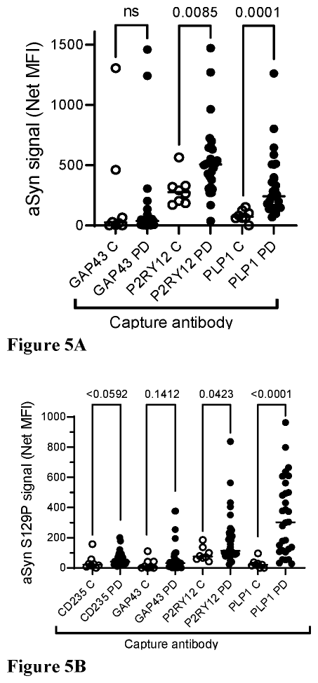

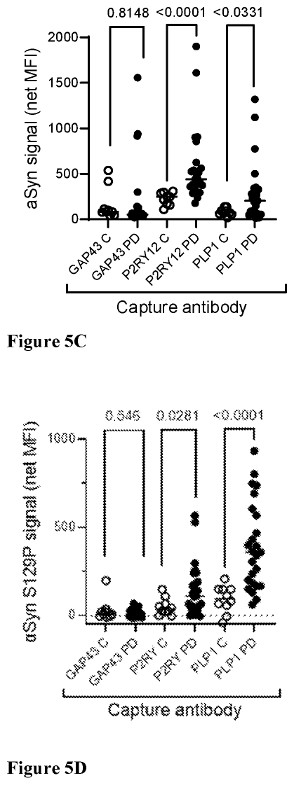

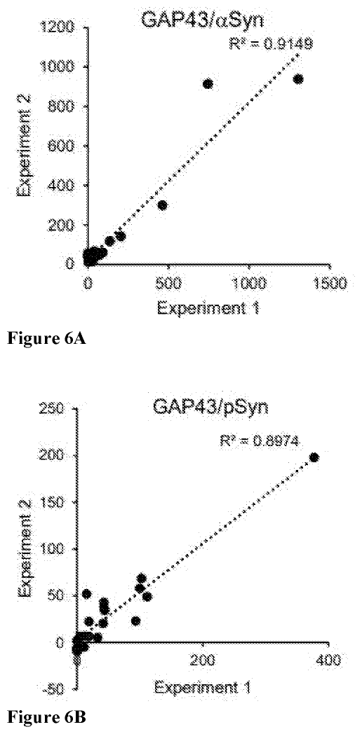

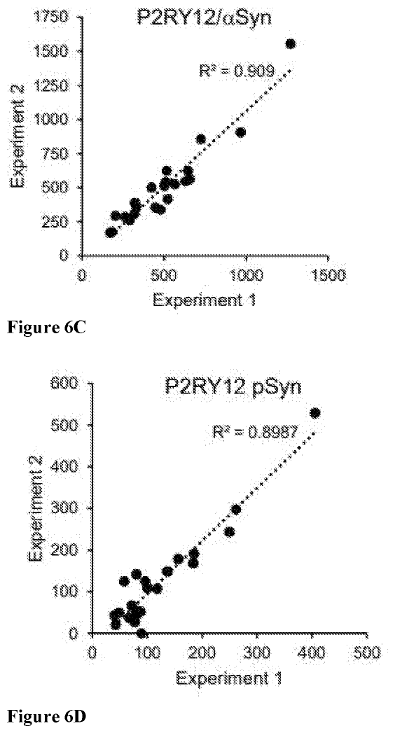

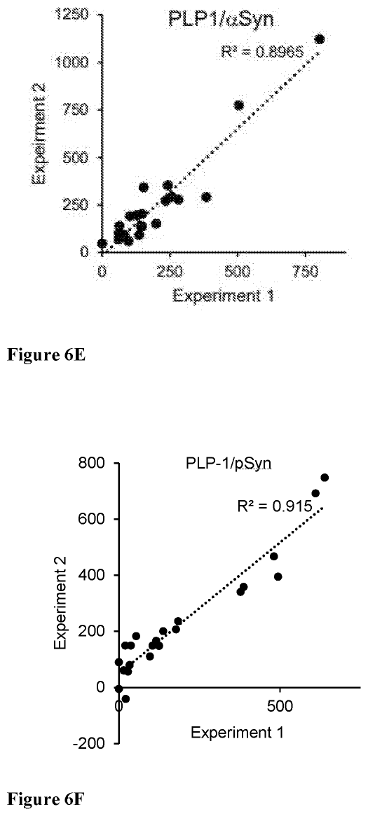

Lastly, the reproducibility of the assay was examined by independent analysis of two aliquots of the same PD and controls plasma samples. The results for the first and second experiments are shown in FIGS. 5A-5B (first experiment, αSyn and αSyn S129P, respectively) and 5C-5D (second experiment, αSyn and αSyn S129P, respectively), and the correlations between the two experiments, analyzed for all six outcomes, are plotted in FIGS. 6A-6F. As can be seen, the significant differences between PD and controls were reproduced in the second experiment as well, with high correlation level between the signal in the first and second analyses. Specifically, as can be seen in FIG. 6A-6F, the lowest Pearson correlation coefficient measured was 0.88, indicating remarkable reproducibility.

In contradistinction, other assays, such as commercially available ELISA kits (based on traditional sandwich analysis of two antibodies against two different epitopes within the alpha-synuclein protein), failed to yield significant or strong separation between plasma of PD patients and controls. Examples of such assays are meso-scale (K151WKP-1), SIMOA (HD-1) or ELISA (KHB0061).

Thus, the assays disclosed herein demonstrate linear, dilution-dependent signal reduction, low sample input requirement (25 μl or lower), specificity to exosomes and to αSyn, high intra-assay precision and reproducibility between assays, thereby exhibiting remarkable and unexpected compatibility for clinical-grade analyses.

### Example 5. Measurement of Surface-Bound αSyn Levels in EVs From Plasma of PD, LBD and AD Patients

The developed Luminex assay was used to measure αSyn on the surface of EVs derived from neurons, microglia and oligodendrocyte in plasma samples of subjects with various synucleinopathies: Parkinson's Disease (PD), Lewy Body Disease (LBD) and mixed Alzheimer's Disease (AD) pathology, which is a sub-population of AD characterized by mixed pathologies.

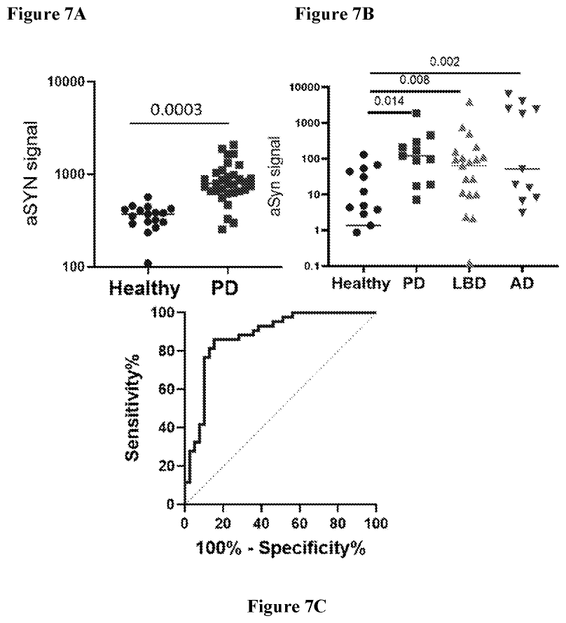

Preparation and characterization of intact exosome Luminex (IEL) beads was carried out as described in Example 1 above, with capture antibodies against GAP43, PLP1 and P2RY12. Capture and detection were performed as described in Example 1. The level of αSyn on the surface of the three central nerve system EV types was summed together. The results are summarized in FIGS. 7A-7C.

FIG. 7A shows the results for a cohort of 32 PD patients and 17 age matched healthy controls. As can be seen in the figure, a significant increase of surface αSyn is observed in plasma samples of PD patients compared to the healthy controls.

FIG. 7B shows the results for a cohort of 12 healthy controls, 11 PD patients, 18 LBD patients, and 11 AD patients. As can be seen in the figure, the level of surface αSyn is significantly higher in all three synucleinopathies than in the healthy controls. It is important to note that cohorts presented in A and B were run separately.

The results of PD and healthy controls from both cohorts were harmonized into a single ROC curve, presented in FIG. 7C, which shows the potential of the method to identify PD with 84% sensitivity and 78% specificity.

The foregoing description of the specific embodiments will so fully reveal the general nature of the invention that others can, by applying current knowledge, readily modify and/or adapt for various applications such specific embodiments without undue experimentation and without departing from the generic concept, and therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

1-13. (canceled)

14. A method of analyzing extracellular vesicle (EV) populations in a sample of a subject, the method comprising:
a. providing a capture system, comprising at least three populations of distinct fluorescence-labeled magnetic microspheres, wherein each microsphere population displays antibodies directed to distinct targets on the surface of distinct neural and/or glial EV populations,
b. providing 1-75 μl of a non-processed blood-derived sample of the subject, or a corresponding amount of intact EV;
c. incubating the sample with the capture system, under conditions such as to allow specific antigen-antibody binding while substantially maintaining the integrity of the EV membranes, to thereby provide distinct populations of EV-microsphere complexes corresponding to each target;
d. washing the EV-microsphere complexes using a magnetic device, under conditions enabling selective capturing of said complexes;
e. incubating the captured complexes with at least one labeled detection antibody, the antibody directed to a neuronal or glial membrane-bound α-synuclein, under conditions such as to allow specific antigen-antibody binding while substantially maintaining the integrity of the EV membranes;
f. washing the resulting labeled complexes using a magnetic device to remove excess reagents;
g. subjecting the resulting complexes to a microfluidic device amenable for simultaneously detecting and quantifying fluorescent emission on a plurality of wave lengths, to thereby quantify the fluorescence emission levels and provide a separate assessment of the α-synuclein level corresponding to each of the EV populations; and
h. comparing the assessed levels to control levels;
wherein the method is performed using reagents and under conditions so as to retain said EV in a substantially intact form.

15. The method of claim 14, wherein the sample is a plasma or serum sample.

16. The method of claim 15, wherein 1-50 μl of plasma or serum are provided.

17. The method of claim 14, wherein said sample is obtained from a subject afflicted with, or suspected of having, a synucleinopathy, or a synucleinopathy-associated condition.

18. The method of claim 17, wherein the synucleinopathy is selected from the group consisting of Parkinson's disease (PD), Lewy body dementia (LBD), PD with dementia (PDD), pure autonomic failure (PAF), multiple system atrophy (MSA) and mixed Alzheimer's disease (AD) pathology.

19. The method of claim 14, further comprising diagnosing or evaluating a synucleinopathy selected from the group consisting of PD, LBD, PDD, PAF, MSA and mixed AD pathology, in said subject, wherein αSyn levels that are significantly higher than the levels corresponding to a healthy control subject, indicate the presence of a synucleinopathy in said subject, and/or wherein an αSyn levels that are not substantially higher than the levels corresponding to a healthy control subject, indicate the absence of a synucleinopathy in said subject.

20. (canceled)

21. The method of claim 17, wherein the subject is diagnosed with, or is suspected of having, a dementia or cognitive decline.

22. The method of claim 21, wherein αSyn levels that are significantly higher than the levels corresponding to a healthy control subject indicate the presence of a dementia or cognitive decline associated with αSyn pathology.

23. The method of claim 18, further comprising determining treatment for said subject, wherein determining treatment comprises determining that said subject in amenable for treatment with one or more agents indicated for management of a synucleinopathy or a condition associated therewith, or wherein determining treatment comprises determining that said subject in not amenable for treatment with one or more agents that are contraindicated for, or excluded from management of, a synucleinopathy or a condition associated therewith.

24-25. (canceled)

26. The method of claim 23, wherein the subject is determined to be afflicted with a dementia or cognitive decline associated with αSyn pathology, and the method comprises determining that said subject is not amenable for treatment with one or more agents selected from the group consisting of: anticholinergic drugs, dopamine precursors, dopamine agonists, and first-generation antipsychotics (FGA).

27. The method of claim 23, further comprises further comprising treating the subject determined to be afflicted with the synucleinopathy or synucleinopathy-associated condition with one or more agents indicated for management of said synucleinopathy or a condition associated therewith.

28. The method of claim 14, further comprising selecting the at least one labeled detection antibody to be used in step e. as an antibody capable of selectively identifying αSyn on the surface of an EV populations of a neuronal or glial origin, and not on an erythrocyte-derived EV (EDE) population.

29. The method of claim 28, wherein said antibody is capable of selectively identifying αSyn on the surface of an GAP43, PLP-1 and/or P2RY12-displaying EV populations, and not on a CD235a-displaying EV population.

30. A method for determining the compatibility of an assay or reagent for the diagnosis of a synucleinopathy, comprising assessing the EV selectivity of the assay or reagent, wherein if said assay or reagent is determined to be capable of selectively identifying αSyn on the surface of an EV populations of a neuronal or glial origin, and not on an erythrocyte-derived EV (EDE) population, said assay or reagent is determined to be compatible with the diagnosis of a synucleinopathy.

31. The method of claim 30, wherein said assay or reagent comprises an antibody.

32. The method of claim 31, wherein said antibody is capable of selectively identifying αSyn on the surface of an GAP43, PLP-1 and/or P2RY12-displaying EV populations, and not on a CD235a-displaying EV population.

33. A kit for evaluating or diagnosing a synucleinopathy, comprising:
i) a capture system, comprising a first population of magnetic microspheres displaying an antibody directed to GAP43, and labeled by a first combination of fluorophores, a second population of magnetic microspheres displaying an antibody directed to PLP-1, and labeled by a second combination of fluorophores, and a third population of magnetic microspheres displaying an antibody directed to P2RY12, and labeled by a third combination of fluorophores;
ii) at least one detection antibody capable of selectively identifying αSyn on the surface of an EV populations of a neuronal or glial origin, and not on an erythrocyte-derived EV (EDE) population; and optionally
iii) reagents for performing said evaluation under conditions so as to retain said EV in a substantially intact form.

34. The kit of claim 33, wherein the magnetic microspheres are further coated with negatively-charged peptides amenable for diminishing non-specific interactions, or wherein the at least one detection antibody is fluorescently labeled and is capable of selectively identifying αSyn on the surface of an GAP43, PLP-1 and/or P2RY12-displaying EV populations, and not on a CD235a-displaying EV population.

35. (canceled)

36. The kit of claim 34, wherein the at least one detection antibody is fluorescently labeled by quantum dots or by combinations of multiple fluorophores.

37. The kit of claim 33, wherein the reagents are selected from the group consisting of:
(i) at least one binding buffer for incubating a sample with the capture system to thereby provide distinct populations of EV-microsphere complexes, the at least one binding buffer characterized by lack of detergents and by the presence of protease and/or phosphatase inhibitors;
(ii) at least one washing buffer, characterized by significantly enhanced salt concentrations compared to the at least one binding buffer; and
(iii) at least one binding buffer and at least one washing buffer as defined in (i) and (ii) above.
